(19) United States
(12) Patent Application Publication
(10) Pub. No.: US 2026/0060909 A1
(43) Pub. Date: Mar. 5, 2026

(54) HAIR CARE COMPOSITION COMPRISING TERTIARY AMIDOAMINES SURFACTANT (TAS), SALICYLIC ACID, FREE OF FATTY ALCOHOL AND PH= 6.5

(71) Applicant: L'OREAL, Paris (FR)

(72) Inventors: Su YUAN, Shanghai (CN); Qingsheng TAO, Shanghai (CN)

(73) Assignee: L'OREAL, Paris (FR)

(21) Appl. No.: 19/105,220

(22) PCT Filed: Oct. 28, 2022
(86) PCT No.: PCT/CN2022/128171

§ 371 Date: Feb. 20, 2025

(51) Int. Cl. A61K 8/42; A61K 8/34; A61K 8/368; A61K 8/41; A61K 8/891; A61Q 5/12

(52) U.S. Cl. CPC A61K 8/42; A61K 8/342; A61K 8/368; A61K 8/416; A61K 8/891; A61Q 5/12

(57) ABSTRACT

A composition for conditioning the hair comprises: a) at least one tertiary amidoamine surfactant; b) at least one beta hydroxy acid; and c) optionally a C16-C22 fatty alcohol, wherein the molar ratio of the tertiary amidoamine surfactant to the beta hydroxy acid is from 1:1 to 1:0.01, the pH of the composition is at least 6.5, if presents, the total amount of the C16-C22 fatty alcohol is lower than lwt. %.

### TECHNICAL FIELD

The present invention belongs to the field of cosmetic product. In particular, the present invention relates to a composition for conditioning the hair. The present invention also relates to a cosmetic process for conditioning the hair.

### BACKGROUND ART

The appearance and/or condition of keratin fibers are often affected by both extrinsic and intrinsic factors such as aging.

Keratin fibers, especially hair, are constantly exposed to harsh extrinsic conditions, such as sun, chemical damage, e.g., from detergents, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, skin cells shedded from the scalp, pollution, dirt, and extreme humidity conditions.

The above-described factors can result in thinning hair and/or harm the visual appearance and the feel of the hair, and lead to lusterless body and decreased volume.

For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive.

Furthermore, hair can lose its desirable attributes due to physical stress on the hair such as brushing and application of heat. As such, these factors generally result in damage to the keratin fibers, either by affecting protective materials on the surface of the hair (the cuticle), or by altering the hair fiber internally (the cortex).

Thus, it is highly desirable to have products providing the hair conditioning benefits.

Hair rinse conditioners have conventionally been based on a combination of a cationic surfactant, which is generally a quaternary ammonium compound, and fatty alcohols. This combination results in a gel-network structure which provides the hair smoothness, provides compositions with thick, creamy rheology and will build up on the hair.

Thus, there is a need to formulate a cosmetic composition for conditioning the hair, which can provide the hair smoothness without building up on the hair.

### SUMMARY OF THE INVENTION

An object of the present invention is thus to develop a composition for conditioning the hair, which can provide the hair softness without building up on the hair.

Thus, according to a first aspect, the present invention provides a composition for conditioning the hair comprising:
a) at least one tertiary amidoamine surfactant;
b) at least one beta hydroxy acid; and
c) optionally a C16-C22 fatty alcohol,
wherein
the molar ratio of the tertiary amidoamine surfactant to the beta hydroxy acid is from 1:1 to 1:0.01,
the pH of the composition is at least 6.5,
if presents, the total amount of the C16-C22 fatty alcohol is lower than 1 wt. %.

The composition according to the present invention provides the hair smoothness and softness without building up on the hair.

In addition, the composition according to the present invention is still easily applied due to its dense texture.

Furthermore, in some embodiments, the composition according to the present invention exhibit a pearl-like appearance, which is desired.

According to a second aspect, the present invention provides a process for conditioning the hair comprising applying the composition as described above onto the hair.

Other characteristics and advantages of the invention will emerge more clearly on reading the description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well as optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

As used herein, the expression “at least one” used in the present description is equivalent to the expression “one or more” and may be substituted for it.

According to the first aspect of the present invention, a composition for conditioning the hair comprises:
a) at least one tertiary amidoamine surfactant;
b) at least one beta hydroxy acid; and
c) optionally a C16-C22 fatty alcohol.

### Tertiary Amidoamine Surfactant

According to the first aspect, the composition of the present invention comprises at least one tertiary amidoamine surfactant.

Amidoamines are a class of chemical compounds that are formed from fatty acids and diamines.

Preferably, the tertiary amidoamine surfactant is selected from tertiary amidoamines with a C₁₀-C₃₀ alkyl chain.

More preferably, the tertiary amidoamine surfactant is selected from (C₁₀-C₃₀)alkylamido(C₁-C₈)alkyl(di) (C₁-C₆)alkylamines.

By (C₁₀-C₃₀)alkylamido(C₁-C₈)alkyl (di) (C₁-C₆)alkylamine it must be understood that the alkyl group can be linear or branched, and by amido it must be understood the group —C(O)—N(R)— or —N(R)—C(O)— wherein R represents a hydrogen atom or a, linear or branched, (C₁-C₆)alkyl group.

More preferably, the tertiary amidoamine surfactant is selected from those of the following formula:

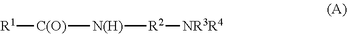

wherein:
R¹ is an alkyl or alkenyl group having a C₁₀-C₃₀ carbon chain,
R² is a linear C₁-C₆ alkylene group, and
R³ and R⁴, same or different, are linear alkyl groups having a C₁-C₄ carbon chain.

Preferably in the formula (A),
R¹ is a linear alkyl group having a C₁₂-C₂₄ carbon chain,
R² is a linear C₁-C₄ alkylene group, and
R³ and R⁴ are same, representing methyl or ethyl groups.

More preferably, in the formula (A), R² is a —(CH₂)₃— group, and R³ and R⁴ are —CH₃ groups.

Non-limiting examples of tertiary amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and combinatios thereof.

Preferably, the tertiary amidoamine surfactant is selected from brassicamidopropyl dimethylamine, stearamidopropyl dimethylamine, behenamidopropyl dimethylamine, and combinations thereof.

In some embodiments, the composition of the present invention comprises brassicamidopropyl dimethylamine.

Brassicamidopropyl dimethylamine can be represented by the following formula (A1):

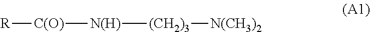

wherein R—C(O) is a fatty acid derived from brassica campestris (rapeseed oil) seed oil, with a majority of behenyl (C₂₂) group.

As brassicamidopropyl dimethylamine useful in the composition of the present invention, mentions maybe made of PROCONDITIONT™ 22 from the company Inolex Chemical Company.

In some embodiments, the composition of the present invention comprises stearylamidopropyl dimethylamine.

Stearylamidopropyl dimethylamine can be represented by the following formula (A2):

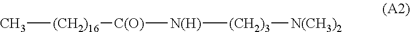

As stearylamidopropyl dimethylamine useful in the composition of the present invention, mentions may be made especially of TEGO® AMID S 18 from Evonik company and GENAMIN® SPA from Clarient company.

Advantageously, the tertiary amidoamine surfactant is present in the composition of the present invention in an amount ranging from 0.1 wt. % to 10 wt. %, preferably from 2 wt. % to 8 wt. %, more preferably from 2.4 wt. % to 5 wt. %, relative to the total weight of the composition.

### Beta Hydroxy Acids

According to the first aspect, the composition of the present invention comprises at least one beta hydroxy acid.

The term “beta-hydroxy acid” is understood to mean, according to the present invention, a carboxylic acid having a hydroxyl functional group and a carboxylic functional group separated by two carbon atoms.

Preferably, the beta hydroxy acid is selected from salicylic acid and its derivative of the formula:

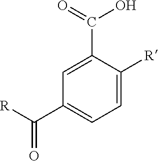

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;
R is a hydroxyl group or an ester functional group of the following formula:

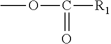

wherein R₁ is a linear or branched saturated or unsaturated aliphatic group having from 1 to 18 carbon atoms.

Preferred salicylic acid derivatives include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid.

More preferably, the beta hydroxy acid is selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof.

Most preferably, the beta hydroxy acid is salicylic acid.

Salicylic acid, or 2-hydroxybenzoic acid, is provided to the chemical peel composition to provide enhanced penetration of the composition into the skin. Salicylic acid penetrates deeper into the skin than alpha hydroxy acids, such as lactic acid.

Advantageously, the beta hydroxy acid is present in the composition of the present invention in an amount ranging from 0.05 wt. % to 1 wt. %, preferably from 0.1 wt. % to 0.5 wt. %, and more preferably from 0.15 wt. % to 0.3 wt. %, relative to the total weight of the composition.

The inventors have found that without fatty alcohols and at a pH condition of at least 6.5, which can be achieved by the molar ratio of the tertiary amidoamine surfactant to the beta hydroxy acid mentioned below, the composition of the present invention forms tighter and denser lamellar structure to deliver improved wet and dry hair smoothness, as compared with traditional conditioner with a lamellar structure. Meanwhile, such tighter and denser lamellar structure can bring unique pearlescent appearance without any additional pearling agent and no “build up” behavior of fatty alcohols which is the common concern of traditional conditioner.

Advantageously, the molar ratio of the tertiary amidoamine surfactant to the beta hydroxy acid is from 1:1 to 1:0.01, preferably from 1:0.6 to 1:0.05, more preferably from 1:0.5 to 1:0.09.

In some embodiments, the composition according to the present invention comprises stearamidopropyl dimethylamine and salicylic acid, the molar ratio of stearamidopropyl dimethylamine to salicylic acid is from 1:1 to 1:0.01, preferably from 1:0.6 to 1:0.05, more preferably from 1:0.5 to 1:0.2.

In some embodiments, the composition according to the present invention comprises brassicamidopropyl dimethylamine and salicylic acid, the molar ratio of brassicamidopropyl dimethylamine to salicylic acid is from 1:1 to 1:0.03, preferably from 1:0.4 to 1:0.06, more preferably from 1:0.2 to 1:0.09.

### Fatty Alcohols

According to the first aspect, the composition of the present invention optionally comprises a C16-C22 fatty alcohol.

If presents, the amount of C16-C22 fatty alcohol in the composition according to the present invention is lower than 1 wt. %.

Preferably, the composition according to the present invention is free of C16-C22 fatty alcohol.

More preferably, the composition according to the present invention is free of fatty alcohol containing more than 8 carbon atoms, preferably more than 10 carbon atoms.

pH

Preferably, the pH of the composition according to the present invention is at least 7.3.

More preferably, the pH of the composition according to the present invention is from 7.3 to 8.

### Additional Cosmetic Active Ingredients

Depend on the final purpose, the composition according to the present invention can comprises one or more additional cosmetic active ingredients.

As cosmetic active ingredients that may be used in the composition of the present invention, examples that may be mentioned include silicones, humectants, cationic conditioners, among others.

The cationic conditioner is not particularly limited. For example, it can be any commonly used in the art, such as cationic surfactants, for example, distearyldimethylammonium chloride, cetyltrimethylammonium chloride, behenyltrimethylammonium chloride, behentrimontum chloride, cetrimonium chloride, oleocetyldimethylhydroxyethylammonium chloride, stearamidopropyldimethyl (myristyl acetate) ammonium chloride, di(C₁-C₂ alkyl) (C₁2-C₂₂ alkyl)hydroxy(C₁-C₂ alkyl)ammonium salt, such as dialkyldimethylammonium or alkyltrimethylammonium salt in which the alkyl radical preferably comprises 12 to 24 carbon atoms, propanetallowdiammonium dichloride, behentrimonium methosulfate, quaternium-87, and combinations thereof.

Cationic polymers such as polymeric quaternary ammonium salts, for example Polyquaternium-7 copolymer of acrylamide and diallyldimethylammonium chloride, Polyquaternium-10 quaternized hydroxyethyl cellulose, Polyquaternium-11 copolymer of vinylpyrrolidone and quaternized dimethylaminoethyl methacrylate, Polyquaternium-22 copolymer of acrylic acid and diallydimethylammonium chloride and combinations thereof, can be used as cationic containers in the composition according to the present invention.

In some embodiments, the composition according to the present invention comprises a cationic conditioner selected from distearyldimethylammonium chloride, cetyltrimethylammonium chloride, behenyitrimethylammonium chloride, behentrimontum chloride, cetrimonium chloride, oleocetyldimethylhydroxyethylammonium chloride, stearamidopropyldimethyl (myristyl acetate) ammonium chloride, di(C₁-C₂ alkyl) (C₁2-C₂₂ alkyl)hydroxy(C₁-C₂ alkyl)ammonium salt, such as dialkyldimethylammonium or alkyltrimethylammonium salt in which the alkyl radical preferably comprises 12 to 24 carbon atoms, propanetallowdiammonium dichloride, behentrimonium methosulfate, quaternium-87, Polyquaternium-7 copolymer of acrylamide and diallyldimethylammonium chloride, Polyquaternium-10 quaternized hydroxyethyl cellulose, Polyquaternium-11 copolymer of vinylpyrrolidone and quaternized dimethylaminoethyl methacrylate, Polyquaternium-22 copolymer of acrylic acid and diallydimethylammonium chloride, and combinations thereof.

The inventors have found that with the presence of cationic surfactant, such as quaternium-87, the composition can deliver both wet hair and dry hair a more softness feeling.

It is easy for the skilled in the art to adjust the amount of the additional cosmetic active ingredient based on the final use of the composition according to the present invention.

### Additional Adjuvants or Additives

The composition according to the present invention may comprise conventional cosmetic adjuvants or additives, for instance, preserving agents and bactericides, hydrophilic thickeners, and fragrance.

The skilled in the art can select the amount of the additional adjuvants or additive so as not to adversely impact the final use of the composition according to the present invention.

### Aqueous Phase

The composition according to the present invention comprises an aqueous phase.

Preferably, the aqueous phase is a continuous phase.

The aqueous phase of the present invention comprises water.

The aqueous phase may also comprise water-miscible organic solvents (at room temperature of 20-25° C.), for instance polyols such as C2-C6 polyols, more particularly glycerin, hexylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, and combinations thereof.

Advantageously, the aqueous phase is present in an amount ranging from 70 wt. % to 99 wt. %, preferably from 80 wt. % to 95 wt. %, relative to the total weight of the composition.

According to a preferred embodiment, the present invention provides a composition for conditioning the hair comprising, relative to the total weight of the composition:
a) from 2.4 wt. % to 5 wt. % of at least one tertiary amidoamine surfactant selected from (C₁₀-C₃₀)alkylamido(C₁-C₈)alkyl(di) (C₁-C₆)alkylamines; and
b) from 0.1 wt. % to 0.5 wt. % of salicylic acid,
wherein
the molar ratio of tertiary amidoamine surfactant to beta hydroxy acid is from 1:0.5 to 1:0.09,
the pH of the composition is at least 7.3,
the composition is free of C16-C22 fatty alcohol.

### Preparation and Use

The composition according to the present invention can be prepared by mixing the components contained therein.

The composition according to the present invention can be used to provide hair conditioning benefits, for example, provide the hair softness without building up on the hair and causing harshness and irritation to the scalp.

The use of the composition can be done on wet or dry hair.

According to the second aspect of the present invention, the present invention provides a process for conditioning the hair comprising applying the composition as described above onto the hair.

The following examples are given by way of illustration of the present invention and shall not be interpreted as limiting the scope.

### EXAMPLES

Main raw materials used, trade names and supplier thereof are listed in Table 1.

TABLE 1

| INCI Name | Trade Name | Supplier |
|---|---|---|
| STEARAMIDORPOPYL | MACKINE 301U | RHODIA (SOLVAY) |
| DIMETHYLAMINE | | |
| BRASSICAMIDOPROPYL | PROCONDITION ™ 22 | INOLEX CHEMICAL |
| DIMETHYLAMINE | | COMPANY |
| SALICYLIC ACID | SALICYLIC ACID | ALTA LABORATORIES |
| CETEARYL ALCOHOL | ECOROL 68/50/MB | ECOGREEN |
| | | OLEOCHEMICALS |
| LACTIC ACID | PURAC FCC 80 | PURAC |
| AMODIMETHICONE | Y-20477 | MOMENTIVE |
| | | PERFORMANCE |
| | | MATERIALS |
| PEG-180 | POLYGLYKOL 8000 S | CLARIANT |
| QUATERNIUM-87 | VARISOFT W 575 PG N | EVONIK |
| | | GOLDSCHMIDT |
| CITRIC ACID | CITRIC ACID | CITRIQUE BELGE |
| | MONOHYDRATE | |
| | GRANULAR | |

### Invention Examples 1-7 and Comparative Examples 1-6

Compositions according to invention examples (IE.) 1-7 and comparative examples 1-6 were prepared with the ingredients listed in Tables 2-4 (the contents are expressed in wt. %):

TABLE 2

| | IE. 1 | IE. 2 | IE. 3 | IE. 4 | IE. 5 |
|---|---|---|---|---|---|
| Components | Wt. % | Wt. % | Wt. % | Wt. % | Wt. % |
| STEARAMIDORPOPYL | 2.4 | 5 | — | — | — |
| DIMETHYLAMINE | | | | | |
| BRASSICAMIDOPROPYL | — | — | 2.6 | 5 | 5 |
| DIMETHYLAMINE | | | | | |
| SALICYLIC ACID | 0.24 | 0.5 | 0.1 | 0.2 | 0.4 |
| CETEARYL ALCOHOL | — | — | — | — | 1 |
| WATER | 97.36 | 94.5 | 97.3 | 94.8 | 93.6 |
| Molar ratio of tertiary | 1:0.27 | 1:0.27 | 1:0.11 | 1:0.11 | 1:0.23 |
| amidoamine surfactant | | | | | |
| to beta hydroxy acid | | | | | |
| pH | 7.34 | 7.34 | 7.69 | 7.69 | 7.77 |

TABLE 3

| | CE.1 | CE. 2 | CE. 3 | CE. 4 | CE. 5 | CE. 6 |
|---|---|---|---|---|---|---|
| Components | Wt. % | Wt. % | Wt. % | Wt. % | Wt. % | Wt. % |
| STEARAMIDOPROPYL DIMETHYLAMINE | 2.67 | — | 5 | | — | — |
| BRASSICAMIDOPROPYL DIMETHYLAMINE | — | 2.92 | | 5 | 5 | 5 |
| SALICYLIC ACID | 1 | 1 | — | — | 0.2 | 0.4 |
| CETEARYL ALCOHOL | — | — | — | — | — | 3 |
| LACTIC ACID | — | — | — | — | 0.56 | — |
| CITRIC ACID | — | — | 0.52 | 0.52 | — | — |
| WATER | 96.33 | 96.08 | 94.48 | 94.48 | 94.24 | 91.6 |
| Molar ratio of tertiary amidoamine surfactant | 1:1 | 1:1 | 1:0.2 | 1:0.22 | 1:0.11 | 1:0.23 |
| to beta hydroxy acid | | | | | | |
| pH | 5.35 | 6.44 | 6.84 | 6.94 | 6.04 | 7.61 |

TABLE 4

| | IE. 6 | IE. 7 |
|---|---|---|
| Components | Wt. % | Wt. % |
| BRASSICAMIDOPROPYL | 5 | 5 |
| DIMETHYLAMINE | | |
| AMODIMETHICONE | 1 | 1 |
| SALICYLIC ACID | 0.2 | 0.34 |
| PEG-180 | — | 1.5 |
| QUATERNIUM-87 | — | 2.15 |
| WATER | 93.8 | 90.01 |
| Molar ratio of tertiary amidoamine | 1:0.11 | 1:0.19 |
| surfactant to beta hydroxy acid | | |
| pH | 7.70 | 7.73 |

Compositions of invention examples 1-7 are compositions according to the present invention.

Compositions of comparative examples 1, 2, and 5 have a pH below 6.5.

Compositions of comparative examples 3 and 4 comprise an alpha-hydroxyl acid rather than a beta hydroxy acid.

Composition of comparative example 5 comprises 3 wt. % of cetearyl alcohol.

### Preparation Procedure:

The compositions above were prepared as follows, taking composition of invention example 1 as an example:
1) adding salicylic acid and water into a main vessel, and heating to 80° C. with gentle mixing until salicylic acid fully dissolved to get a clear liquid;
2). heating stearamidorpopyl dimethylamine to 80° C. to melt into a liquid;
3). adding the melted stearamidorpopyl dimethylamine into the main vessel at 80° C. and homogenizing for 15 minutes;
4). cooling the main vessel to 30° C. with gentle mixing to obtain the composition.

### Evaluation

The appearance and texture of compositions of invention examples 1-6 and comparative examples 1-6 were observed.

Compositions of invention examples 1 and 2 comprise stearamidopropyl dimethylamine and salicylic acid with the same molar ratio of stearamidopropyl dimethylamine to salicylic acid. Composition of invention example 1 shows lotion texture with strong pearling appearance. Composition of invention example 2 shows cream texture with strong pearling appearance.

Compositions of invention examples 3 and 4 comprise brassicamidopropyl dimethylamine and salicylic acid with the same molar ratio of brassicamidopropyl dimethylamine to salicylic acid. Composition of invention example 3 shows lotion texture with strong pearling appearance. Composition of invention example 4 shows cream texture with strong pearling appearance.

Compositions of invention example 1 to 4 demonstrate the impact of the absolute amount of the tertiary amidoamine surfactant in the composition on the final texture.

Composition of invention example 5 comprises brassicamidopropyl dimethylamine, salicylic acid and cetearyl alcohol. Composition of invention example 5 shows cream texture with pearling appearance.

Compositions of comparative examples 1 and 2 comprise stearamidopropyl dimethylamine/brassicamidopropyl dimethylamine and salicylic acid with the molar ratio of tertiary amidoamine surfactant to salicylic acid out of the claimed range. Compositions of comparative examples 1 and 2 show clear liquid texture without pearling appearance.

Composition of comparative example 3 comprises stearamidopropyl dimethylamine and citric acid. It shows white heterogeneous thin cream texture. Composition of comparative example 4 comprises brassicamidopropyl dimethylamine and citric acid. It shows phase separation texture.

Composition of comparative example 5 comprises brassicamidopropyl dimethylamine, salicylic acid and lactic acid. It shows phase separation texture.

Composition of comparative example 6 comprises brassicamidopropyl dimethylamine, salicylic acid and cetearyl alcohol. It shows white cream without pearling appearance.

Compositions of invention examples 1-5 have denser lamellar structure than composition of comparative example 6. And other comparative examples cannot form lamellar structures.

In addition, compositions of invention examples 1-5 exhibit a pearl-like appearance, while compositions of comparative examples 1-6 do not exhibit a pearl-like appearance.

Hair Wet and Dry Sensory Hair wet and dry sensory tests were carried out on 6 g China natural hair (length: 27 cm, diameter: 2.5 cm) dyed twice with standard process for invention examples and comparative examples.

### Wet Hair Sensory

First, the hair swatch was treated as follows.
1. using 2.4 g shampoo without silicone to prewash the hair swatch, massaging the hair swatch for 1 minute;
2. rinsing off the hair swatch with water at 36.5° C. with fixed flow rate for 30 seconds;
3. extruding water using two fingers with once;
4. applying 2.4 g sample to be evaluated onto the prewashed hair swatch, and massaging for 1 minute;
5. rinsing off the hair swatch with water at 36.5° C. with fixed flow rate for 30 seconds;
6. extruding water using two fingers with once and hanging up the hair swatch.

Then, six panelists were invited to give scores for two testing samples from −2′ to 2′ with progressive increasing score of 0.25′. The scores of sensory attributes of benchmark were normalized to zero. Based on this marking standard, the score located within ±0.5˜±0.75 shows slight difference. The score being equal or greater than ±1 indicates significant difference.

### Dry Hair Sensory

Above steps 1-6 were repeated, then the hair swatch was leaved at room temperature for dry.

Then, six panelists were invited to give scores for the two testing samples from −2′ to 2′ with progressive increasing score of 0.25′. The scores of sensory attributes of benchmark were normalized to zero. Based on this marking standard, the score located within ±0.5±0.75 shows slight difference. The score being equal or greater than ±1 indicates significant difference.

Table 5 shows the evaluation results of compositions of invention examples 1-5. It can be seen from Table. 5 that as compared with the benchmark composition of comparative example 6, compositions of invention examples 2, 4 and 5 deliver a significantly better smoothness and slightly improved softness with significant less coating on both wet hair and dry hair.

TABLE 5

| | Sensory | | | | | |
|---|---|---|---|---|---|---|
| | Wet hair | | | Dry hair | | |
| | smooth- | soft- | | smooth- | soft- | |
| Examples | ness | ness | coating | ness | ness | coating |
| CE. 6 | 0 | 0 | 0 | 0 | 0 | 0 |
| (benchmark) | | | | | | |
| IE. 1 | N/A (lotion like, low viscosity) | | | | | |
| IE. 2 | 1.5 | 0.75 | −1.5 | 1 | 1.25 | −1 |
| IE. 3 | N/A (lotion like, low viscosity) | | | | | |
| IE. 4 | 1.25 | 0.75 | −2 | 1 | 0.5 | −1.25 |
| IE. 5 | 1 | 0.5 | −1 | 1 | 0.5 | −1 |

Table 6 shows the results derived from the softness scores of compositions of invention example 6 and 7.

TABLE 6

Sensory

| Examples | Wet softness | Dry softness |
|---|---|---|
| IE. 6 | 0 | 0 |
| (benchmark) | | |
| IE. 7 | 1.25 | 1 |

1: A composition for conditioning the hair, comprising:
a) a tertiary amidoamine surfactant;
b) a beta hydroxy acid; and
c) optionally a C16-C22 fatty alcohol,
wherein
a molar ratio of the tertiary amidoamine surfactant to the beta hydroxy acid is from 1:1 to 1:0.01,
the pH of the composition is at least 6.5, and
if present, a total amount of the C16-C22 fatty alcohol is less than 1 wt. %.

2: The composition according to claim 1, wherein the tertiary amidoamine surfactant is a (C₁₀-C₃₀)alkylamido(C₁-C₈)alkyl(di)(C₁-C₆)alkylamine.

3: The composition according to claim 1, wherein the tertiary amidoamine surfactant is of formula (A) 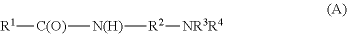
wherein:
R¹ is an alkyl or alkenyl group having a C₁₀-C₃₀ carbon chain,
R² is a linear C₁-C₆ alkylene group, and
R³ and R⁴ are each independently linear alkyl groups having a C₁-C₄ carbon chain.

4: The composition according to claim 1, wherein the tertiary amidoamine surfactant is selected from the group consisting of oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl, tallamidopropyl brassicaamidopropyl dimethylamine, olivamidopropyl dimethylamine, palmitamidopropyl dimethylamine, stearamidoethyldiethylamine, and combinations thereof.

5. The composition according to claim 1, wherein the tertiary amidoamine surfactant is selected from the group consisting of brassicamidopropyl dimethylamine, stearamidopropyl dimethylamine, behenamidopropyl dimethylamine, and combinations thereof.

6: The composition according to claim 1, wherein the tertiary amidoamine surfactant is present in an amount of from 0.1 wt. % to 10 wt. % relative to the total weight of the composition.

7: The composition according to claim 1, wherein the beta hydroxy acid is selected from salicylic acid and a compound of the formula: 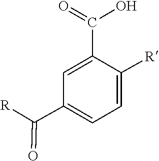
wherein R is a linear, branched or cyclic saturated aliphatic group or an aliphatic unsaturated group containing one or a number of double bonds, which may or may not be conjugated, these groups containing from 2 to 22, carbon atoms and being able to be substituted by at least one substituent selected from the group consisting of a halogen atom, a trifluoromethyl group, a hydroxyl group a hydroxyl group esterified by an acid having from 1 to 6 carbon atoms, a carboxyl functional group, and a carboxyl group esterified by a alcohol having from 1 to 6 carbon atoms;
R′ is a hydroxyl group or an ester functional group of the formula: 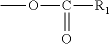
wherein R₁ is a linear or branched saturated or unsaturated aliphatic group having from 1 to 18 carbon atoms.

8: The composition according to claim 1, wherein the beta hydroxy acid is present in an amount of from 0.05 wt. % to 1 wt. % relative to the total weight of the composition.

9: The composition according to claim 1, wherein the pH of the composition is 7.3 or higher.

10: The composition according to claim 1, comprising, relative to the total weight of the composition:
a) from 2.4 wt. % to 5 wt. % of at least one tertiary amidoamine surfactant selected from (C₁₀-C₃₀)alkylamido(C₁-C₈)alkyl(di)(C₁-C₆)alkylamines; and
b) from 0.1 wt. % to 0.5 wt. % of salicylic acid,
wherein
the molar ratio of the at least one tertiary amidoamine surfactant to the beta hydroxy acid is from 1:0.5 to 1:0.09,
the pH of the composition is at least 7.3, and
the composition is free of a C16-C22 fatty alcohol.

11: The composition of claim 1, further comprising a cosmetic active ingredient selected from the group consisting of a silicone, a humectant; a cationic conditioner, and combinations thereof.

12: The composition of claim 11, wherein the cationic conditioner is present and is selected from the group consisting of distearyldimethylammonium chloride, cetyltrimethylammonium chloride, behenyitrimethylammonium chloride, behentrimontum chloride, cetrimonium chloride, oleocetyldimethylhydroxyethylammonium chloride, stearamidopropyldimethyl (myristyl acetate) ammonium chloride, di(C₁-C₂ alkyl)(C₁₂-C₂₂ alkyl)hydroxy(C₁-C₂ alkyl)ammonium salt, propanetallowdiammonium dichloride, behentrimonium methosulfate, quaternium-87, Polyquaternium-7 copolymer of acrylamide and diallyldimethylammonium chloride, Polyquaternium-10 quaternized hydroxyethyl cellulose, Polyquaternium-11 copolymer of vinylpyrrolidone and quaternized dimethylaminoethyl methacrylate, Polyquaternium-22 copolymer of acrylic acid and diallydimethylammonium chloride, and combinations thereof.

13: The composition of claim 1, which is free of a C16-C22 fatty alcohol.

14: A process for conditioning the hair, comprising: applying the composition of claim 1 onto the hair.
